(19) United States
(12) Patent Application Publication
(10) Pub. No.: US 2010/0099981 A1
(43) Pub. Date: Apr. 22, 2010

(54) Trans-Septal Catheterization Device And Method

(76) Inventor: Robert S. Fishel, Delray Beach, FL (US)

Correspondence Address: MCHALE & SLAVIN, P.A., 2855 PGA BLVD, PALM BEACH GARDENS, FL, 33410, US

(21) Appl. No.: 12/552,329

(22) Filed: Sep. 2, 2009

Related U.S. Application Data
(60) Provisional application No. 61/107,219, filed on Oct. 21, 2008.

(51) Int. Cl. A61B 6/00

(52) U.S. Cl. 600/424

(57) ABSTRACT

A method and apparatus for performing medical treatment on a patient. The method include generating a three dimensional image of a portion of a patient and overlaying a real time image of a treatment tool in the patient during its movement in the patient to a site of interest.

### RELATED APPLICATION

This application claims the benefit under 35 U.S.C. 119 (e) of U.S. Provisional Patent Application No. 61/107,219, filed Oct. 21, 2008, entitled, “Method And Device For Safe Transeptal Cardiac Catheterization And Other Anatomically Guided Tissue Punctures”, the entirety of which is incorporated herein by reference.

### FIELD OF INVENTION

A device is provided for use in surgical procedures such as the treatment of atrial fibrillation. The device involves a catheter having an end or functional portion locatable in three dimensions in real time.

### BACKGROUND OF THE INVENTION

Certain surgical procedures like cardiac catheterization for the treatment of atrial fibrillation utilize catheters that need to be precisely placed in three dimensional space in a patient to reduce the risk of patient injury and to improve the chances of conducting a successful medical treatment procedure. One of the problems is that the use of procedures to view the patient's interior in real time, like fluoroscopy, cannot be used continuously for long periods of time which poses problems for its use in long medical procedures. Additionally, fluoroscopy cannot always provide distinction or high contrast between tissue portions because there is little if any contrast between parts in a patient in the visual output. Fluoroscopy also provides only a two dimensional image. Other viewing devices are available to provide improved imaging, but are not real time, like CT scans that utilize x-ray as an energy source. Magnetic resonance has been recommended as providing an image in more real time than fluoroscopy. Another problem with heart procedures is that the heart moves during beating and positions of the various parts relative to one another change with time.

Trans-septal heart procedures are sometimes favored to provide a surgical pathway from the right atrium to the left atrium, the right atrium being a preferred entry point to the heart. This procedure is particularly difficult to perform because of the need to precisely place the puncture needle on the septum at the foramen ovalis or fossa ovalis which is three dimensional while the fluoroscope provides an image in only two dimensions. After the puncture is made, a pathway for tools is provided between the left and right atriums. Additionally, if the needle goes too far during the puncture process, it can injure or puncture the opposing wall of the left atrium or other parts of the heart and vascular system. Such damage can result in the procedure not being performed or major injury to the patient. While there are risks attendant with such puncture procedures, their value as a treatment regimen outweighs the risks. However improvements in the performance of such procedures is desirable to reduce the risks and improve the accuracy of the treatments.

### SUMMARY

The present invention involves the provision of a medical procedure that tracks the movement of a surgical tool in the patient in real time using a previously created three dimensional image of the area of interest. The method includes creating a three dimensional first image of an area of interest of a patient and identifying a fiducial point in the patient for referencing and registering the position and movement of the tool. The tool has a trackable reference point and is inserted into the patient. The reference point is located relative to the fiducial point and its 3-D position is registered for use on the first image. The tool is moved in the patient and location data for the reference point is generated during movement of the tool from the fiducial point in the patient in real time. A second image of at least a portion of the tool is created and is overlaid on the first image to create a third image in three dimensions to show the location of the tool relative to the area of interest in the patient in real time. A medical procedure, such as trans-septal ablation utilizing the tool can be performed by a surgeon utilizing the third image.

The present invention also involves the provision of a system for conducting a medical procedure, the system includes a position sensing apparatus operable to sense the location, and preferably the orientation of a tool in three dimensions in real time in a patient. A data transfer device is coupled to the position sensing apparatus and is operable to transfer data indicative of the position of a reference point on a surgical tool and preferably orientation of the tool. A data processing system is operable to receive data from the data transfer device and assign a location to the reference point and preferably tool orientation. The data processing system also includes a memory operable for storing data indicative of a three dimensional image of an anatomical portion of a patient. The data processing system is programmed to combine the location of the reference point with the stored data to produce data for creating a combined image of the reference point location registered on the three dimensional image. A display device is operably connected to the data processing system and is operable to display the combined image.

### BRIEF DESCRIPTION OF THE DRAWINGS

FIG. 1 is a schematic illustration of a system usable in the present invention to display an image of a tool portion and a 3-D image of an area of interest in a patient.

Like numbers used throughout this application represent like or similar parts and/or construction.

### DETAILED DESCRIPTION OF THE INVENTION

While the present invention is susceptible of embodiment in various forms, there is shown in the drawings and will hereinafter be described presently preferred embodiments with the understanding that the present disclosure is to be considered an exemplification of the invention and is not intended to limit the invention to the specific embodiments illustrated.

In the conduct of medical procedures on a patient, it is desired to know the location of the area of interest for treatment or manipulation and the location of a tool or a medically functional portion of the tool like a distal end of a needle. In some procedures, this is done by physically exposing the area of interest of the patient and the surgeon or other medical personnel directly observing the treatment procedure. However, in some procedures, the treatment area and operative procedure cannot be directly observed. One such procedure is heart ablation to treat cardiac arrhythmia which is done inside the heart with a catheter. Because of blood in the heart, a camera cannot be used inside the heart for real time viewing. Fluoroscopy can be used to provide real time images, but presents problems, it provides two dimensional images while the areas of interest need to be treated positionally in three dimensions. Additionally, some of the treatments can last for hours making exposure of the patient and medical personnel to the radiation a problem and thus prohibitive. Real time images in three dimensions are not possible using 3-D imaging techniques like CT scans and MRI. CT scans also use x-rays limiting their usefulness.

The present invention will be described in terms of it use to perform trans-septal heart ablation, however, it is to be understood that it can be used to perform other medical procedures including tests. The invention provides for three dimensional guidance of surgical device such as a needle for the purposeful puncture of a wall of the heart or to provide access to other generally inaccessible area of the heart.

A three dimensional (3-D) image data set of an area of interest is generated prior to the medical treatment. The data set can be utilized to generate a, so called, 3-D image on a display device such as a monitor. A 3-D image is an approximate representation, on a flat surface (such as monitor), of an image as it is perceived generally by the eye. Two types of such images include perspective and isometric images, commonly used graphical projections by draftsmen and artists. While the displayed image is not truly 3-D because it is displayed on a two dimensional screen it is perceived as 3-D since the image has attributes of depth and preferably can be manipulated or rotated for orientation to show perspective and is herein defined and designated as 3-D. True 3-D image displays are available and are also included in the definition of 3-D image. The 3-D image is computer generated and can be considered virtual. The display apparatus is preferably adapted to rotate the image to show different perspectives of an area of interest, for example, the septum, the ostia of the coronary sinus, the ostia of the left main coronary artery and the like. Such imaging and systems are well known in the art and provide detailed, high contrast and detailed images. The image data is input into a memory for later processing, manipulation and display from a desired selected perspective.

For the medical treatment, the patient is suitably prepared and positioned. A suitable surgical tool, such as a Brockenbrough needle and sheath are selected to perform catheterization. The tool is introduced into an appropriate vein and moved into the heart. The point of introduction can be in the groin, neck or other suitable area of the patient. Such a procedure is referred to as a percutaneous procedure. The tool is provided with a reference point that can be tracked from outside the patient. One method of tracking is through the use of magnetic fields as is done in electroanatomical mapping procedures. Such mapping uses external magnetic fields or changes in impedance to track an object in the body in 3-D space with currently available technology allowing for determination of coordinates on X, Y and Z axes and also pitch and yaw of the field sensor. The field sensor would be utilized as a reference point on the tool and is preferably positioned adjacent the distal end of the tool or surgically functional part of the tool, such as a needle tip. The point of interest on the tool, such as the distal end can be positionally registered to the reference point for tracking and display as later described. Current magnetic field detector technology allows for magnetic field sensor construction in the sub-millimeter space resulting in the ability to provide a field detector located within, upon or integrated into a needle for determining the position and/or orientation of the needle according to a magnetic field generated in the vicinity of the needle tip. The sensor can be the reference point and located adjacent the tool portion of interest for performing the medical procedure such as a needle tip. Field detectors are known and may include a ferromagnetic core and a winding around the core. The winding is connected to a signal receiver and is operable to produce a signal that can be interpreted by the receiver to indicate position and orientation. The output signal can be processed and utilized by a data processing device such as a digital computer with a memory. The location of the sensor (reference point) and at least an image of a portion of the tool can be displayed in real time overlaid or imposed on the previously obtained three dimensional image of the anatomical area of interest to create a combined image to virtually display the tool portion and anatomical area of interest in three dimension positionally registered relationship to assist the surgeon in guiding the tool to the desired area. Encoders can also be used to track movement of the reference point and tool movement and orientation.

At the initial stage of the treatment and after the tool is inserted into the patient, the tool has a portion of it placed at a fiducial point to register its initial position and orientation relative to the fiducial point. It is preferred that the operative portion of the tool, like the needle tip, be placed at the fiducial point. A virtual image of the tool or tool portion is then overlaid onto the 3-D image in registered location relationship for viewing by a surgeon or the like. The combined images will show the tool in position relative to the anatomical structure of the patient. The surgeon may change the perspective of the 3-D image to provide a desired combined image. The movement and position of the tool are shown in real time. The tool is manipulated by the surgeon to reach a desired point for use, for example, the foramen ovalis (or fossa ovalis) of the atrial septum. A puncture is made through the septum to provide an opening between the left atrium and the right atrium. Because of the image being displayed to the surgeon in real time, precise location of the puncture can be accomplished while any close tissue can be identified to prevent it from being negatively contacted by the needle to prevent unintended damage. The puncture can then be used as a pathway for wires, ablation devices and the like to perform the medical procedure.

By selecting the correct fiducial point and accurately modeling the geometry of the tool relative to the reference point, the desired accuracy of tool placement can be achieved. When generating the anatomical data for the 3-D image, care should be taken since the heart is moving during the anatomical image data generation.

More than one anatomical fiducial point may be selected and utilized during the treatment procedure including tool insertion into the patient. This would allow for tracking of the tool during insertion into the anatomical area of interest. Registering the reference points at each of the fiducial points can be performed during the insertion and beginning of use of the tool. Additional fiducial points can be identified and utilized for tool position and orientation registration after insertion of the tool, e.g., at the septum.

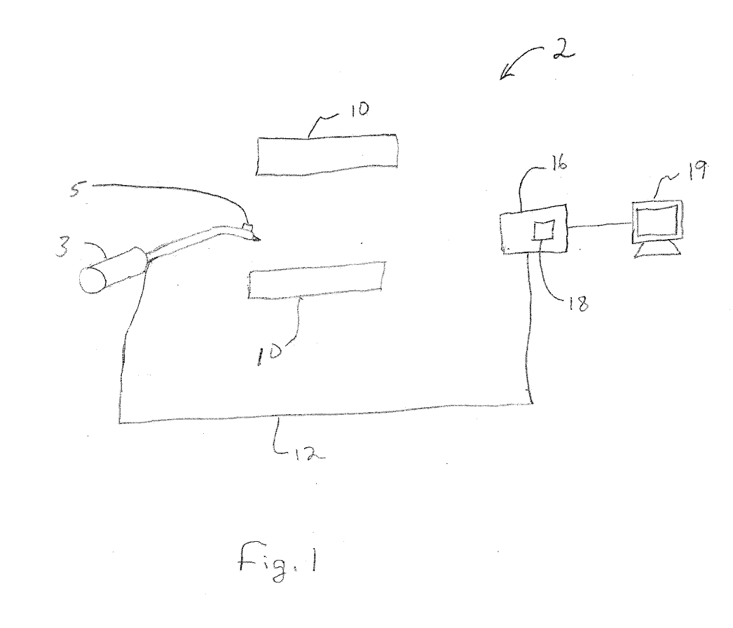

FIG. 1 illustrates a system 1 for conducting a medical procedure. The system includes a position sensing apparatus 2 operable to sense the location of a medical tool 3 such as a catheter in three dimensions in real time. The tool 3 has a reference point 5 that may be a field sensor as described above. The tool 3 can be or include a needle portion with a field sensor mounted thereto. The apparatus 2 may be an electroanatomical mapping device that utilizes magnetic fields from magnets 10 that are sensed by the field sensor to provide a signal to a data transfer device 12 coupled to the field sensor. The field sensor is utilized as a position sensing apparatus and in one embodiment is operable to generate a location and tool orientation signal. The data transfer device 12 may be a cable that is operable to transfer signal data from the sensor that is indicative of the position of a reference point on the tool 3. While the position and preferably also the orientation of the sensor is determined, the position of one or more parts of the tools may also be provided. This can be done by providing the shape and size geometry of the tool as data in a data processing system 16. The data processing system 16 is operable to receive data from the data transfer device 12 and assign a 3-D location to the reference point as well as orientation of the sensor and hence tool 3. The data processing system 16 includes a memory 18 operable for storing data indicative of the three dimensional image of an anatomical portion, such as a heart, of a patient. The 3-D image data in input into the memory for later recall to generate an image on a display device 19, such as a monitor. The data processing system is programmed to combine the location of a reference point and the 3-D image stored data to produce data for creating a combined image of the reference point location positionally registered on the three dimensional image. The display device 19 is operably connected to the data processing system 16 and operable to display the combined image. Both elements of the combined image are preferably virtual.

It is to be understood that while certain forms of the invention are illustrated and described, the invention is not to be limited to the specific forms or arrangements herein described and shown. It will be apparent to those skilled in the art that various changes may be made without departing from the scope of the invention and the invention is not to be considered limited to what is shown and described in the specification and any drawings/figures included herein.

One skilled in the art will readily appreciate that the present invention is well adapted to carry out the objectives and obtain the ends and advantages mentioned, as well as those inherent therein. The embodiments, methods, procedures and techniques described herein are presently representative of the preferred embodiments, are intended to be exemplary and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

What is claimed is:

1. A method of conducting a surgical procedure on a patient, the method including:
creating a three dimensional first image of an area of interest of a patient;
identifying a fiducial point in the patient;
inserting a surgical tool into the patient, the surgical tool having a trackable reference point;
locating the reference point relative to the fiducial point;
generating location data for the reference point during movement of the tool from the fiducial point in the patient and tracking the tool movement in real time;
creating a second image of at least a portion of the tool and overlaying the second image on the first image to create a third image in three dimensions to show the location of the tool relative to the area of interest in the patient in real time; and
performing a medical procedure utilizing the tool.

2. The method of claim 1 wherein the second image being virtual.

3. The method of claim 2 wherein the third image being virtual.

4. The method of claim 2 wherein the locating of the reference point relative to the fiducial point being done with fluoroscopy.

5. The method of claim 1 wherein the medical procedure involving treating the heart.

6. The method of claim 5 wherein the medical procedure including ablation.

7. The method of claim 5 wherein the medical procedure including puncturing the septum.

8. The method of claim 1 wherein the tracking of the reference point using magnetic fields.

9. The method of claim 1 wherein the reference point being tracked using encoders.

10. The method of claim 1 wherein the fiducial point including an ostia.

11. The method of claim 1 wherein the orientation of the tool being tracked in real time and being overlaid on and displayed on the first image.

12. A method of conducting a surgical procedure on the heart, the method including:
creating a three dimensional first image of the heart;
inserting a tool into the body of a patient with a portion of the tool being positioned adjacent the heart, said tool having a first trackable reference point;
establishing a first fiducial point in the heart;
making the first image available to a surgeon;
locating the first reference point relative to the first fiducial point and registering the location of at least a portion of the tool on the first image;
displaying in real time a registered second image of at least a portion of the tool on the first image to create a third image and displaying the third image in three dimensions;
moving the tool relative to the first fiducial point and generating location data for the first reference point and displaying a 3-D registered image of the tool overlaid on the first image to show its location in the patient substantially in real time and three dimensionally; and
guiding the tool to a site of interest in the heart by utilizing the location of the first reference point.

13. A system for conducting a medical procedure, the system including:
a position sensing apparatus operable to sense the location of a tool in three dimensions in real time;
a data transfer device coupled to the position sensing apparatus and operable to transfer data indicative of the position of a reference point on a surgical tool;
a data processing system operable to receive data from the data transfer device and assign a location to the reference point, said data processing system also including a memory operable for storing data indicative of a three dimensional image of an anatomical portion of a patient, said data processing system being programmed to combine the location of the reference point with the stored anatomical data to produce data for creating a combined image of the reference point location registered on the three dimensional image; and
a display device operably connected to the data processing system and operable to display the combined image.
